(19) United States
(12) Patent Application Publication
(10) Pub. No.: US 2006/0068008 A1
(43) Pub. Date: Mar. 30, 2006

(54) Lyophilized pharmaceutical compositions

(76) Inventors: Ketan Amin, Randolph, NJ (US); Rose-Marie Dannenfelser, Bloomfield, NJ (US); Joseph Zielinski, Florham Park, NJ (US); Barbara Wang, Berkeley Heights, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/230,193

(22) Filed: Sep. 19, 2005

Related U.S. Application Data
(60) Provisional application No. 60/612,976, filed on Sep. 24, 2004.

(51) Int. Cl. A61K 9/20

(52) U.S. Cl. 424/464

(57) ABSTRACT

Pharmaceutical compositions that include a poorly water-soluble therapeutic compound, an aqueous solvent, a nonvolatile cosolvent and a bulking agent. The pharmaceutical compositions can be orally ingested or administered parenterally. The pharmaceutical compositions can further be lyophilized to form a pharmaceutically acceptable cake that can be administered orally, e.g., as a solid oral dosage form; or reconstituted and administered parenterally, e.g. as a single i.v. bolus.

### FIELD OF THE INVENTION

The present invention relates to lyophilized pharmaceutical compositions and the process of manufacture thereof.

### BACKGROUND OF THE INVENTION

Lyophilization, or more commonly known as freeze-drying, is a process which extracts water from a solution to form a granular solid or powder. The process is carried out by freezing the solution and subsequently extracting any water or moisture by sublimation under vacuum.

As compared to other drying techniques, lyophilization offers many advantages. For example, the quality of the substance being lyophilized is preserved while reducing the total weight of that substance. Furthermore, degradation of the therapeutic compound in a drug product is minimized since the lyophilized material is no longer exposed to water and air (especially when sealed in a vial that had been purged with a non-reactive gas such as nitrogen or argon); thus, the shelf life of the therapeutic compound is lengthened and enhanced. Additionally, lyophilized pharmaceutical compositions typically do not require particular conditions, such as refrigeration, for storage. Lyophilization is particularly useful for developing pharmaceutical drug products that are reconstituted and administered to a patient by injection, for example parenteral drug products. Alternatively, lyophilization is useful for developing oral drug products, especially fast melts or flash dissolve formulations.

Many new therapeutic compounds exhibit poor aqueous solubility. To make such active pharmaceutical ingredients suitable for administration, e.g., parenterally, additional solubilizing excipients are often added. Often these poorly water-soluble therapeutic compounds are incorporated into systems that contain water and an organic solvent, called a cosolvent system. Although these liquid cosolvent systems increase solubility, they may do little to augment the stability of the therapeutic compound. As a result, lyophilization of these cosolvent systems provides for a beneficial way of enhancing both physical and chemical stability of the therapeutic compound.

Typically, the ideal lyophilization medium has a high vapor pressure, a melting point either below or slightly above room temperature (about 25° C.), low toxicity and should be rapidly and completely removed to produce a stable and readily reconstitutable cake. Solubility enhancers, typically used in cosolvent systems include propylene glycol, polyethylene glycols, and polysorbate 80. However, prior attempts to lyophilize cosolvent systems have focused primarily on excipients, such as organic solvents with relatively high vapor pressure, e.g., ethanol, isopropanol, or tert-butanol, to ensure removal of the solvent from the pharmaceutical composition. Such excipients have potential disadvantages that include toxicity, operator safety due to the high degree of flammability or explosivity, lack of commercial grades or monographs, requirements of special manufacturing facilities/equipment and/or storage areas, difficult handling properties, requirements of high-purity solvents, minimal residual solvent levels in the final composition, high usage cost, potential for splash/spattering of the product in the vial neck and lack of regulatory familiarity.

Thus, there is a need for a cosolvent system that minimizes the aforementioned disadvantages while maintaining characteristics that allow the pharmaceutical composition to be suitable for lyophilization. Additionally, the resulting lyophilized cake possesses pharmaceutically acceptable properties.

### SUMMARY OF THE INVENTION

The present invention relates to a pharmaceutical composition comprising a therapeutic compound (especially a poorly water-soluble therapeutic compound), an aqueous solvent, i.e., water, a nonvolatile cosolvent and a bulking agent. In a particular embodiment of the present invention, the nonvolatile cosolvent comprises less than or equal to thirty percent weight/volume (w/v) of the composition. Additionally, the bulking agent comprises less than or equal to five percent (w/v) of the composition. In one aspect of the invention, a pharmaceutically acceptable cake resulting from the lyophilization of the pharmaceutical composition is described. In another aspect of the invention, the pharmaceutical composition is a pharmaceutically acceptable cake resulting from the lyophilization of the aforementioned solution. After this cake is reconstituted a solution is once again obtained; this solution is acceptable for parenteral administration, e.g., administered as an intravenous (i.v.) bolus dose; or oral administration, e.g., a drink. The pharmaceutically acceptable cake itself can be formed into a solid oral dosage form, e.g., a fast-melt or flash-dissolve tablet.

In another aspect of the present invention, the pharmaceutical composition contains a liquid polyethylene glycol (PEG) as the nonvolatile cosolvent, i.e., any PEG that is in a liquid state at room temperature and pressure and a solid PEG as the bulking agent, i.e., any PEG that is in a solid state at room temperature and pressure. Such a system does not require any other cosolvents, especially volatile cosolvents, like lower alkyl, i.e., C₁-C₄, alcohols and bulking agents.

In a further aspect of the present invention, a process for making a pharmaceutically acceptable cake that can be reconstituted with water for parenteral administration is disclosed. This process comprises the steps of forming a solution comprising a therapeutic compound, especially a poorly water-soluble therapeutic compound, an aqueous solvent, i.e., water; a nonvolatile cosolvent, e.g., a liquid PEG; and a bulking agent, e.g., a solid PEG; and lyophilizing the solution to form a pharmaceutically acceptable cake.

### DETAILED DESCRIPTION OF THE INVENTION

The present invention relates to a pharmaceutical composition that is suitable for parenteral or oral administration that comprises a therapeutic compound, an aqueous solvent, i.e., water; a nonvolatile cosolvent; and a bulking agent. The present invention also relates to the pharmaceutically acceptable cake that results form the freeze-drying of the pharmaceutical composition. The pharmaceutically acceptable cake can be administered orally or parenterally after reconstitution, or swallowed orally. In addition to the aforementioned components, the solution can also optionally contain other excipients, such as buffers, pH adjusters, stabilizers, surfactants and other adjuvants recognized by one of ordinary skill in the art to be appropriate for such a composition. Examples of such excipients are described in Handbook of Pharmaceutical Excipients, 4^{th} Edition, Rowe et al., Eds., Pharmaceutical Press (2003).

As used herein, the term “pharmaceutical composition” means a solution containing a therapeutic compound to be administered to a mammal, e.g., a human. A pharmaceutical composition is “pharmaceutically acceptable” which refers to those compounds, materials, compositions and/or dosage forms, which are, within the scope of sound medical judgment, suitable for contact with the tissues of mammals, especially humans, without excessive toxicity, irritation, allergic response and other problem complications commensurate with a reasonable benefit/risk ratio.

As used herein, the term “therapeutic compound” means any compound, substance, drug, medicament or active ingredient having a therapeutic or pharmacological effect, and which is suitable for administration to a mammal, e.g., a human. Such therapeutic compounds should be administered in a “therapeutically effective amount”.

As used herein, the term “therapeutically effective amount” refers to an amount or concentration which is effective in reducing, eliminating, treating, preventing or controlling the symptoms of a disease or condition affecting a mammal. The term “controlling” is intended to refer to all processes wherein there may be a slowing, interrupting, arresting or stopping of the progression of the diseases and conditions affecting the mammal. However, “controlling” does not necessarily indicate a total elimination of all disease and condition symptoms, and is intended to include prophylactic treatment.

The appropriate therapeutically effective amount is known to one of ordinary skill in the art as the amount varies with the therapeutic compound being used and the indication which is being addressed. For example in accordance with the present invention, the therapeutic compound may be present in amount less than or equal to 10% (w/v).

The pharmaceutical composition or pharmaceutically acceptable cake, as described in detail below, will suitably contain between 0.1 mg and 100 mg of the therapeutic compound per unit dose, e.g., 0.1 mg, 1 mg, 5 mg, 10 mg, 20 mg, 25 mg, 50 mg or 100 mg per unit dose.

As used herein, the term “unit dose” means a single dose which is capable of being administered to a subject, and which can be readily handled and packaged, remaining as a physically and chemically stable unit dose comprising the therapeutic compound.

Therapeutic compounds that are particularly suited for the present invention are those that are poorly soluble in water. As used herein, the term “poorly water-soluble” refers to having a solubility in water at 20° C. of less than 1%, e.g., 0.01% (w/v), i.e., a “sparingly soluble to very slightly soluble drug” as described in Remington, The Science and Practice of Pharmacy, 19^{th} Edition, A. R. Gennaro, Ed., Mack Publishing Company, Vol. 1, p. 195 (1995).

Examples of therapeutic classes of therapeutic compounds include, but are not limited to, antihypertensives, antianxiety agents, anticlotting agents, anticonvulsants, blood glucose-lowering agents, decongestants, antihistamines, antitussives, antineoplastics, beta (β)-blockers, anti-inflammatories, antipsychotic agents, cognitive enhancers, anti-atherosclerotic agents, cholesterol reducing agents, antiobesity agents, autoimmune disorder agents, anti-impotence agents, antibacterial and antifungal agents, hypnotic agents, antibiotics, anti-depressants, anti-Parkinsonism agents, anti-Alzheimer's disease agents, antiviral agents and combinations of the foregoing.

The therapeutic compound(s) is present in the pharmaceutical compositions of the present invention in a therapeutically effective amount or concentration. Such a therapeutically effective amount or concentration is known to one of ordinary skill in the art as the amount or concentration varies with the therapeutic compound being used and the indication which is being addressed. For example, in accordance with the present invention, the therapeutic compound may be present in an amount by weight of up to about 20% by weight of the pharmaceutical composition, e.g., from about 0.05% by weight. The therapeutic compound may also be present in an amount from about 0.5-15% by weight of the pharmaceutical composition, e.g., from about 1.5% to about 5% by weight of the pharmaceutical composition.

A therapeutically effective amount of a therapeutic compound is mixed with an aqueous solvent, i.e., water, a nonvolatile cosolvent and a bulking agent to form a solution. The solution contains, e.g., a concentration of the nonvolatile cosolvent from about 0.01% to about 30% (w/v), e.g., about 0. 1% to about 20%, e.g., about 1% to about 10%. Furthermore, the solution contains, e.g., a concentration of the bulking agent from about 0.01% to about 5% (w/v), e.g., 1% to about 3%. Optionally, a surfactant can also be added. The resulting solution is, e.g., homogeneous and optically clear. The solution does not comprise any solvents having a relatively high vapor pressure, e.g., lower alkyl(C₁-C₄) alcohols, such as ethanol, isopropanol or tert-butanol.

As used herein, a nonvolatile cosolvent refers to a substance having a vapor pressure lower than 0.50 mm Hg at 25° C. The purpose of the nonvolatile cosolvent is to facilitate the dissolution of a poorly water-soluble therapeutic compound in water in order to form a solution. Without the presence of the nonvolatile cosolvent, a solution of the poorly water-soluble therapeutic compound does not form. A solution is necessary to form a homogeneous lyophile.

Examples of a nonvolatile cosolvent include, without limitation, alkylene glycols such as, PEG, propylene glycol, polyhydric alcohols, e.g., mannitol, sorbitol and xylitol; polyoxyethylenes; linear polyols, e.g., ethylene glycol, 1,6-hexanediol, neopentyl glycol and methoxypolyethylene glycol; and mixtures thereof.

Particularly useful as a nonvolatile cosolvent is PEG which is the polymer of ethylene oxide that conforms generally to the formula H(OCH₂CH₂)ₙOH, in which n represents the average molecular weight (m.w.) of the polymer.

The types of PEG useful in the present invention can be categorized by its state of matter, i.e., whether the substance exists in a solid or liquid form at room temperature and pressure. As used herein, “liquid PEG” refers to PEG having a m.w. such that the substance is in a liquid state at room temperature and pressure. For example, PEG with an average m.w. less than 800. Particularly useful are PEG 400 (m.w. from about 380420), PEG 600 (m.w. from about 570-630) and mixtures thereof. PEGs are commercially-available from Dow Chemical (Danbury, Conn.) under the CARBOWAX SENTRY line of products.

As used herein, “solid PEG” refers to PEG having a molecular weight such that the substance is in a solid state at room temperature and pressure. For example, PEG having an average m.w. ranging between 900 and 10,000 is a solid PEG. Particularly useful solid PEGs are those having a m.w. between 3,350 (m.w. from about 3015 to about 3685) and 8,000 (m.w. from about 7,000 to 9,000). Especially useful as a solid PEG are PEG 3350, PEG 8000 and mixtures thereof.

In the present invention, the solid PEGs function as bulking agents in the pharmaceutical composition. As used herein, the term “bulking agent” refers to an ingredient that provides bulk to the pharmaceutical composition. Examples of “non-PEG bulking agents” include, without limitation, mannitol, trehalose, lactose, sucrose, polyvinyl pyrrolidone, sucrose, glycine, cyclodextrins, dextran and derivatives and mixtures thereof. Particularly useful as bulking agents are crystalline solids, e.g., solid PEGs.

Surfactants can also be optionally used in the pharmaceutical composition. Surfactants include, but are not limited to, fatty acid and alkyl sulfonates; benzethanium chloride, e.g., HYAMINE 1622 from Lonza, Inc. (Fairlawn, N.J.); polyoxyethylene sorbitan fatty acid esters, e.g., the TWEEN Series from Uniqema (Wilmington, Del.); and natural surfactants, such as sodium taurocholic acid, 1-palmitoyl-2-Sn-glycero-3-phosphocholine, lecithin and other phospholipids. Such surfactants, e.g., minimize aggregation of lyophilized particles during reconstitution of the product. These surfactants may comprise from about 0.01% to about 5% w/v.

Once mixed, the solution is filled into a container that is suitable for lyophilization, e.g., a glass vial. The lyophilization cycle typically includes the following steps: a freezing step, a primary drying step and a secondary drying step.

In the freezing step, the solution is cooled. The temperature and duration of the freezing step is chosen such that all of the ingredients in the composition are completely frozen. For example, a suitable freezing temperature is approximately −40° C. The water in the formulation becomes crystalline ice. The balance of the formulation in the frozen state may be crystalline, amorphous or a combination thereof.

In the primary drying step, the ice formed during freezing is removed by sublimation at sub-ambient temperatures (although greater than the freezing temperature) under vacuum. For example, the chamber pressure used for sublimation can be from about 40 milliTorr to 400 milliTorr and the temperature be between −30° C. to −5° C. During the primary drying step, the formulation should be maintained in the solid state below the collapse temperature (“T_{c}”) of the formulation. The T_{c} is the temperature above which the freeze-dried cake loses macroscopic structure and collapses during freeze-drying. For amorphous products the glass transition temperature (“T_{g}”) or for crystalline products the eutectic temperature (“Tₑ”) are approximately the same as T_{c}. In addition, the T_{g} for the maximally freeze concentrated solution (“T′_{g}”) is important to the development of lyophilization cycles because this represents the highest temperature that is safe for the composition for primary drying.

After primary drying, any residual amounts of liquid which could not be removed by sublimation is removed by secondary drying, i.e., desorption. The temperature during secondary drying is near or greater than ambient temperature.

After lyophilization, the pharmaceutical composition becomes a cake. Such a cake should be pharmaceutically acceptable. As used herein, a “pharmaceutically acceptable cake” refers to a non-collapsed solid drug product remaining after lyophilization that has certain desirable characteristics, e.g., pharmaceutically acceptable, long-term stability, a short reconstitution time, an elegant appearance and maintenance of the characteristics of the original solution upon reconstitution. The pharmaceutically acceptable cake can be solid, powder or granular material. The pharmaceutically acceptable cake may also contain up to five percent water by weight of the cake.

During the lyophilization process, neither the nonvolatile cosolvent nor bulking agent will sublime from the pharmaceutical composition. In the final pharmaceutically acceptable cake, the cake, e.g., comprises from about 0% to about 90% (w/w) of nonvolatile cosolvent; e.g., from about 5% to about 80% (w/w); e.g., from about 10% to about 70%; e.g., from about 20% to about 60% (w/w). Furthermore, the cake, e.g., comprises from about 10% to about 80% (w/w) of the bulking agent; e.g., from about 20% to about 70% (w/w); e.g., from about 30% to about 60% (w/w).

Multiple experiments are conducted using PEG 400 as the nonvolatile cosolvent in combination with various commonly used bulking agents. Table 1 lists the T′_{g} for the common bulking agents alone and with varying concentrations of PEG 400.

TABLE 1

| | % PEG 400 | | |
|---|---|---|---|
| Bulking agent | 0% | 10% | 20% |
| PEG 8000 5% | −67° C. | −77° C. | −79° C. |
| Mannitol 5% | −29° C. | −81° C. | −80° C. |
| Sucrose 10% | −33° C. | −71° C. | −75° C. |
| PVP K90 5% | −21° C. | −82° C. | not tested |

The addition of up to 20% PEG 400 causes a negligible shift in the T′_{g} for an aqueous solution of 5% PEG 8000. Conversely, addition of PEG 400 to solutions of mannitol, sucrose or PVP results in a significant decrease in T′_{g}. This significant decrease in T′_{g} adversely affects the lyocycle development of the respective mixtures. Surprisingly, the addition of PEG 400 to the PEG 8000 solution causes a negligible shift in the eutectic temperature, thus, allowing for the development of a pharmaceutically acceptable cake. Without being bound to a particular theory, it is believed that the crystallization of PEG 8000 is inhibited by PEG 400 upon co-lyophilization. Model crystalline bulking materials, mannitol and PEG 8000, and model amorphous bulking materials, sucrose and PVP, are evaluated with PEG 400 for their interactions in aqueous solutions.

To examine the lyophilization process of various solutions, approximately 3 μL of solution is placed onto the cooling state of a freeze-drying microscope, covered with a quartz slide, and cooled from room temperature to −70° C. at 10° C./min. After a three-minute hold, the samples are reheated to −30° C. at 5° C./min. and then to −18° C. at 1° C./min. After a five-minute hold, the samples are then re-cooled to −35° C. at 10° C./min. and then re-heated to −18° C. at 1° C./min. followed by a return to −35° C. to allow for additional lyophilization along the freeze-drying front. Freeze-drying microscopy observations of 5% PEG 8000 with 10% PEG 400 indicates a T_{c} of −18° C. Similar observations are made by freeze-drying microscopy for 5% PEG 8000 with 20% PEG 400. However, freeze-drying microscopy studies indicated that lyophilization of PEG 400 with mannitol, sucrose, PVP are not feasible at temperatures above −50° C. because of structure collapse or lack thereof within the lyophilized sample.

Modulated differential scanning calorimetery (MDSC) profiles of frozen solutions containing PEG 8000, PEG 400 and their mixtures are evaluated to determine phase behavior during freezing. Frozen aqueous solutions containing PEG 8000, and PEG 400 show a single region between their individual T′_{g}s, indicating that a single phase is formed when frozen.

Thermal analysis of a 5% PEG 8000 aqueous solution yields an endothermic event at −11.18° C. which corresponds to the T_{c}. Upon addition of 10% or 20% PEG 400, this endotherm shifts from −11° C. to −17° C. or −18° C., respectively. Observations of T_{c}s obtained from freeze-drying microscopy correspond to the endothermic events observed in MDSC thermograms for 5% PEG 8000 with 10% or 20% PEG 400. Similarly, thermal analysis of 5% PEG 8000 with 10% or 20% PEG 600 yields endothermic events at −15° C. or −16° C., respectively. The physical state of the solutes in frozen solutions is, e.g., evaluated by MDSC which, in addition, provides an estimate of the maximum allowable product temperature for primary drying which also corresponds to T_{c}.

A T_{g} is not detected by MDSC upon lyophilization of PEG 8000. Thermally stimulated current spectrometry studies, beneficial for materials with low amorphous content, are performed based on methods in Venkatesh et al., Pharm Res, Vol.18, pp. 98-103 (2001) and Boutonnet-Fagegaltier et al., J Pharm Sci, Vol. 91, pp.1548-1560 (2002), which are each herby incorporate by reference in their entirety. Thermally stimulated current analysis indicated that lyophilized PEG 8000 has a T_{g} at −16.16° C. Lyophilization of PEG 8000 with increasing concentrations of PEG 400 resulted in a T_{g} close to the T_{g} of neat PEG 400. The lack of a T_{g} between the T_{g} values of the pure components indicates that lyophilization of PEG 8000 with PEG 400 results in a phase-separated system analogous to immiscible amorphous systems.

Lyophiles, e.g., have contiguous systems of channels or pores created by the sublimation of ice as water vapor travels from the ice to the outer surface of the cake. The porous nature of lyophiles aids in the reconstitution time and therefore is another property necessary for the acceptance of a pharmaceutically acceptable cake. The resultant PEG 8000 and PEG 400 lyophile, because of its porous nature, reconstitutes in approximately less than two minutes, indicative of a good cake with minimal agitation.

In addition to the above experiments, additional studies are conducted to determine whether certain systems containing particular bulking agents in combination with various cosolvents resulted in a pharmaceutically acceptable cake after lyophilization.

Each of the solutions of the following studies are made using the following process:

In a suitable mixing container, the liquid PEG is added. While stirring the liquid PEG, the bulking agent is added. The temperature is increased to about 30-50° C. as needed in order to dissolve the bulking agent. Once dissolved, the solution is cooled to room temperature. Water is then added. The composition is stirred until a clear, homogeneous solution is obtained. The solution is then filled into ten mL serum vials with a two mL fill volume. The vials are transferred to a freeze dryer, e.g., Model SHM 90 from Usifroid (Maurepas, France). The solutions are then cooled to a shelf temperature of about −50° C. at 1° C. minute and held for 0.5 days. A vacuum is then established with primary drying at −25° C. for two days, and secondary drying at 25° C. for 1.2 days.

Table 2 sets forth results of the additional studies.

TABLE 2

| Sample | Bulking agent | | |
|---|---|---|---|
| No. | (w/v %) | Cosolvent (w/v %) | Lyophilized Cake |
| 1 | Citric acid (5%) | None | No cake |
| 2 | | PEG 400 (10%) | Solution |
| 3 | | PEG 400 (10-30%) | Solution |
| 4 | Dextran (5%) | None | Shrinkage |
| 5 | | PEG 400 (10-30%) | Collapse |
| 6 | FICOLL 400* (5%) | None | Good |
| 7 | | PEG 400(10-30%) | Collapse |
| 8 | Glycine (5%) | None | Good |
| 9 | | PEG 400 (10-30%) | Collapse, brown |
| 10 | | PEG 600 (10-30%) | Collapse, brown |
| 11 | HPβCD** (5%) | None | Good |
| 12 | | PEG 400 (10-30%) | Solution |
| 13 | Mannitol (5%) | None | Good |
| 14 | | PEG 200 (10-20%) | Collapse |
| 15 | | PEG 400 (10%) | Center rose up |
| | | | in vial |
| 16 | | PEG 400 (20-40%) | Collapse |
| 17 | | PEG 600 (10%) | Center rose up |
| | | | in vial |
| 18 | | PEG 600 (20-30%) | Collapse |
| 19 | | PEG 1000 (10-20%) | Collapse |
| 20 | | PEG 3350 (0-10%) | Good |
| 21 | | PEG 3350 (20%) | Collapse |
| 22 | | PEG 8000 (10-20%) | Collapse |
| 23 | PEG 600 (10%) | None | Good |
| 24 | PEG 1000 (5%) | None | Good |
| 25 | | PEG 400 (10-40%) | Collapse |
| 26 | | PEG 600 (10-40%) | Collapse |
| 27 | PEG 3350 (2%) | PEG 600 (8%) | Good |
| 28 | PEG 3350 (4%) | PEG 600 (6%) | Good |
| 29 | PEG 3350 (5%) | None | Good |
| 30 | | PEG 400 (10-30%) | Good |
| 31 | | PEG 400 (40%) | Collapse |
| 32 | | PEG 600 (10-30%) | Good |
| 33 | | PEG 600 (40%) | Collapse |
| 34 | PEG 3350 (6%) | PEG 600 (4%) | Good |
| 35 | PEG 8000 (2%) | PEG 600 (8%) | Good |
| 36 | PEG 8000 (4%) | PEG 600 (6%) | Good |
| 37 | PEG 8000 (5%) | PEG 400 (0-30%) | Good |
| 38 | | PEG 400 (10-20%) | Good |
| | | and TBA (5%) | |
| 39 | | PEG 400 (35-40%) | Collapse |
| 40 | | PEG 600 (10-30%) | Good |
| 41 | | PEG 600 (10-30%) | Good |
| | | and TBA (5%) | |
| 42 | | PEG 600 (40-60%) | Collapse |
| 43 | | PEG 600 (40-60%) | Collapse |
| | | and TBA (5%) | |
| 44 | | PEG 600 and | Collapse |
| | | PLURONIC | |
| | | F68*** (5%) | |
| 45 | | PLURONIC F68 (5%) | Good |
| 46 | PEG 8000 (5-10%) | None | Good |
| 47 | PEG 8000 (6%) | PEG 600 (4%) | Good |
| 48 | PEG 8000 (8%) | PEG 600 (2%) | Good |
| 49 | PVP (5%) | None | Shrinkage |
| 50 | | PEG 400 (10-30%) | Solution |
| 51 | Sucrose (10%) | PEG 200 (10-20%) | Solution |
| 52 | | PEG 400 (0-40%) | Collapse |
| 53 | | PEG 400 (30%) | Solution |
| 54 | | PEG 600 (10-20%) | Collapse |
| 55 | | PEG 1000 (20%) | Collapse |
| 56 | | PEG 3350 (20%) | Collapse |
| 57 | | PEG 8000 (20%) | Collapse |

*A hydrophilic polymer of sucrose, available from Serva Electrophoresis GmbH (Heidelburg, Germany).
**Hydroxypropyl β-cyclodextrin.
***A poloxamer, ethylene oxide/propylene oxide block copolymer from BASF (Mt. Olive, NJ).

As shown in the above table, conventional bulking agents dextran, sucrose (FICOLL 400), HPβCD and mannitol (Samples 4, 6, 8,11 and 13, respectively) with the exception of citric acid (Sample 1) form pharmaceutically acceptable cakes after lyophilization provided no nonvolatile cosolvent is added to the solution. Without a nonvolatile cosolvent, it may be difficult, if not impossible, to form a solution containing a poorly water-soluble therapeutic compound.

However, once PEG 400 (a liquid PEG) is added as a cosolvent for dextran, sucrose and glycine (Samples 5, 7, 9 and 10) the cakes formed from lyophilization subsequently collapse, thus, not pharmaceutically acceptable. For HPβCD, and PVP (Samples 12 and 50) the addition of PEG 400 prevents a cake from even forming, the solution prior to lyophilization remains a solution after lyophilization.

The presence of a liquid PEG in a solution containing mannitol in most cases does not form a pharmaceutically acceptable cake (Samples 14-19 and 21-22). PEG 3350, however, does form a pharmaceutically acceptable cake with 5% mannitol (Sample 20)

The presence of a liquid PEG in a solution containing sucrose (Samples 51-57). results in either no formation of a lyophile or a collapsed cake subsequent to freeze drying.

Surprisingly, pharmaceutical compositions comprising a solid PEG, such as PEG 3350 and PEG 8000, form pharmaceutically acceptable cakes even though the solutions contain a liquid PEG, such as PEG 400 and PEG 600. The concentration of the liquid PEG, however, does not exceed 30% (w/v) of the solution. See, results for Samples 23-48.

The following example incorporates a poorly water-soluble therapeutic compound in the pharmaceutical compositions of the present invention. In a suitable mixing container, liquid PEG (PEG 400, PEG 600, or any combination thereof is added. While stirring the liquid PEG, the bulking agent, a solid PEG, (PEG 3350, PEG 8000 or any combination thereof) and diclofenac sodium is added. The temperature is increased to about 30-50° C. as needed in order to dissolve the bulking agent and diclofenac sodium. Once dissolved, the solution is cooled to room temperature. Water is then added. The composition is stirred until a clear, homogeneous solution is obtained. The solution is then filled into ten mL serum vials with a two mL fill volume. The vials are transferred to a freeze dryer, e.g., Usifroid Model SHM 90. The solutions are then cooled to a shelf temperature of about −50° C. at 1° C. per minute and held for 0.5 days. A vacuum is then established with primary drying at −25° C. for two days, and secondary drying at 25° C. for 1.2 days.

It is understood that while the present invention has been described in conjunction with the detailed description thereof that the foregoing description is intended to illustrate and not limit the scope of the invention, which is defined by the scope of the following claims. Other aspects, advantages and modifications are within the scope of the claims.

What is claimed is:

1. A pharmaceutical composition comprising:
(a) a therapeutic compound;
(b) an aqueous solvent;
(c) a nonvolatile cosolvent; and
(d) a bulking agent.

2. The composition of claim 1, wherein said composition further comprises an aqueous solvent.

3. The composition of claim 2, wherein said nonvolatile cosolvent comprises thirty percent or less by weight per volume of the composition.

4. The composition of claim 3, wherein said bulking agent comprises five percent or less by weight per volume of the composition.

5. The composition of claim 1, wherein said therapeutic compound is poorly water-soluble.

6. The composition of claim 1, wherein said bulking agent is a solid polyethylene glycol (PEG).

7. The composition of claim 6, wherein said solid PEG has an average molecular weight ranging between 1000 and 10000.

8. The composition of claim 7, wherein said average molecular weight ranges between 3350 and 8000.

9. The composition of claim 8, wherein said average molecular weight is 8000.

10. The composition of claim 1, wherein said nonvolatile cosolvent is a liquid PEG.

11. The composition of claim 10, wherein said liquid PEG has a molecular weight less than or equal to 800.

12. The composition of claim 2 wherein said composition forms a pharmaceutically acceptable cake after lyophilization.

13. A process of making a pharmaceutically acceptable cake comprising the steps of:
(a) forming a solution comprising a therapeutic compound, an aqueous solvent, a nonvolatile cosolvent and a bulking agent, said solution being free of a volatile solvent; and
(b) lyophilizing said solution to form a pharmaceutically acceptable cake.

14. The process of claim 13, wherein said bulking agent is a solid PEG.

15. The process of claim 14, wherein said solid PEG has an average molecular weight ranging between 1000 and 10000.

16. The process of claim 15, wherein said average molecular weight ranges between 3350 and 8000.

17. The process of claim 15, wherein said average molecular weight is 8000.

18. The process of claim 13, wherein said nonvolatile cosolvent is a liquid PEG.

19. The process of claim 13, wherein said liquid PEG has an average molecular weight less than or equal to 800.

20. The process of claim 13, wherein said solution further comprises a surfactant.

21. The pharmaceutically acceptable cake produced by the process of claim 13.

22. A pharmaceutically acceptable cake comprising:
(a) a poorly water-soluble therapeutic compound;
(b) a nonvolatile cosolvent, said nonvolatile cosolvent comprising from about 5% to about 80% by weight of the cake; and
(c) a bulking agent, said bulking agent comprising from about 10% to about 80% % by weight of the cake.

23. The pharmaceutically acceptable cake of claim 22, wherein said nonvolatile cosolvent is a liquid PEG.

24. The pharmaceutically acceptable cake of claim 23, wherein said bulking agent is a solid PEG.
